(19) United States
(12) Patent Application Publication
(10) Pub. No.: US 2023/0371849 A1
(43) Pub. Date: Nov. 23, 2023

(54) GAIT INFORMATION GENERATION DEVICE, GAIT INFORMATION GENERATION METHOD, AND RECORDING MEDIUM

(71) Applicant: NEC Corporation, Tokyo (JP)

(72) Inventors: Kazuki IHARA, Tokyo (JP); Kenichiro FUKUSHI, Tokyo (JP); Zhenwei WANG, Tokyo (JP); Hiroshi KAJITANI, Tokyo (JP); Fumiyuki NIHEY, Tokyo (JP); Chenhui HUANG, Tokyo (JP); Yoshitaka NOZAKI, Tokyo (JP); Kentaro NAKAHARA, Tokyo (JP)

(73) Assignee: NEC Corporation, Tokyo (JP)

(21) Appl. No.: 18/195,232

(22) Filed: May 9, 2023

(30) Foreign Application Priority Data
May 18, 2022 (JP) 2022-081296

(51) Int. Cl. A61B 5/11

(52) U.S. Cl. CPC A61B 5/112; A61B 2562/0219

(57) ABSTRACT

Provided is a gait information generation device including an acquisition unit that acquires gait data including time-series data of a foot position of a subject, a detection unit that detects a start point and an end point of a gait cycle from the time-series data of the foot position included in the gait data, a calculation unit that calculates a first movement amount component related to the start point and a second movement amount component related to the end point, a gait information generation unit that generates gait information about a gait movement path of the subject using the calculated first movement amount component and the calculated second movement amount component, and an output unit that outputs the generated gait information.

This application is based upon and claims the benefit of priority from Japanese Patent Application No. 2022-081296, filed on May 18, 2022, the disclosure of which is incorporated herein in its entirety by reference.

### TECHNICAL FIELD

The present disclosure relates to a gait information generation device or the like that generates information about gait.

### BACKGROUND ART

With growing interest in healthcare, services that provide information about features included in walking patterns have attracted attention. The feature included in the walking pattern is also referred to as gait. When the behavior during walking can be verified, healthy gait management can be achieved. The information about behavior during walking can be applied to assist of medical diagnosis. For example, when a movement path (also referred to as a gait movement path) for each gait cycle serving as a reference of walking can be estimated, information necessary for diagnosis assistance can be selected according to a behavior change during walking.

Patent Literature 1 (JP 2004 089355 A) discloses a gait motion device used for verification of gait motion. The device of Patent Literature 1 includes a lower limb motion device, a pressure sensor, a storage unit, a comparison arithmetic unit, and a display unit. The pressure sensor is provided in a lower limb motion device in which gait in a biped upright posture is possible. The pressure sensor measures the tread force of both feet. Based on the measurement data stored in the storage unit, the comparison arithmetic unit performs a gravity center calculation during gait from a difference in tread force between both feet. The display unit displays the balance of the center of gravity during gait based on the calculation result by the comparison arithmetic unit.

Patent Literature 2 (JP 5072093 B1) discloses a mobile terminal that determines a traveling direction of a pedestrian. The mobile terminal of Patent Literature 2 includes an acceleration sensor that outputs triaxial acceleration data and a geomagnetic sensor that outputs triaxial geomagnetic data. The mobile terminal of Patent Literature 2 derives a gravity vector in a gravity direction from a plurality of acceleration vectors, and selects a geomagnetic vector related to the gravity vector. In order to convert the gravity vector and the geomagnetic vector of the sensor coordinate system into the world coordinate system, the mobile terminal of Patent Literature 2 calculates a coordinate system transformation matrix in which rotation matrices for respective spatial axes are combined. The mobile terminal of Patent Literature 2 transforms the plurality of acceleration vectors and geomagnetic vectors into the world coordinate system by using the coordinate system transformation matrix. The mobile terminal of Patent Literature 2 calculates, as a direction angle, an angle formed by an approximate straight line representing orthogonal projection of a locus of an acceleration vector group mapped in a world coordinate system onto a ground surface and an axis representing a north position.

In the method of Patent Literature 1, the locus of the balance of the center of gravity in gait is derived according to the difference in the tread force of both feet. However, in the method of Patent Literature 1, although the locus of the balance of the center of gravity is derived, it is not possible to derive the gait movement path for each gait cycle regarding each of both feet.

In the method of Patent Literature 2, a traveling direction of a pedestrian is determined using acceleration data of three axes and geomagnetic data of three axes. However, in the method of Patent Literature 2, although the traveling direction of the pedestrian is determined, it is not possible to derive the gait movement path for each gait cycle regarding each of both feet.

An object of the present disclosure is to provide a gait information generation device and the like capable of generating gait information about a gait movement path for each gait cycle with respect to each of both feet.

### SUMMARY

A gait information generation device according to an aspect of the present disclosure includes an acquisition unit that acquires gait data including time-series data of a foot position of a subject, a detection unit that detects a start point and an end point of a gait cycle from the time-series data of the foot position included in the gait data, a calculation unit that calculates a first movement amount component related to the start point and a second movement amount component related to the end point, a gait information generation unit that generates gait information about a gait movement path of the subject using the calculated first movement amount component and the calculated second movement amount component, and an output unit that outputs the generated gait information.

In a gait information generation method according to an aspect of the present disclosure, the method includes acquiring gait data including time-series data of a foot position of a subject, detecting a start point and an end point of a gait cycle from the time-series data of the foot position included in the gait data, calculating a first movement amount component related to the start point and a second movement amount component related to the end point, generating gait information about a gait movement path of the subject using the calculated first movement amount component and the calculated second movement amount component, and outputting the generated gait information.

A program according to an aspect of the present disclosure causes a computer to execute the steps of acquiring gait data including time-series data of a foot position of a subject, detecting a start point and an end point of a gait cycle from the time-series data of the foot position included in the gait data, calculating a first movement amount component related to the start point and a second movement amount component related to the end point, generating gait information about a gait movement path of the subject using the calculated first movement amount component and the calculated second movement amount component, and outputting the generated gait information.

### BRIEF DESCRIPTION OF THE DRAWINGS

Exemplary features and advantages of the present invention will become apparent from the following detailed description when taken with the accompanying drawings in which:

FIG. 1 is a block diagram illustrating an example of a configuration of a gait information generation device according to a first example embodiment;

FIG. 2 is a conceptual diagram for explaining an example of a gait event in the first example embodiment;

FIG. 3 is a conceptual diagram for explaining an example of a human body surface in the first example embodiment;

FIG. 4 is a conceptual diagram illustrating an example of a gait movement path derived by the gait information generation device according to the first example embodiment;

FIG. 5 is a conceptual diagram illustrating an example of derivation of a gait movement path derived by the gait information generation device according to the first example embodiment;

FIG. 6 is a conceptual diagram illustrating another example of the gait movement path derived by the gait information generation device according to the first example embodiment;

FIG. 7 is a conceptual diagram illustrating another example of derivation of the gait movement path derived by the gait information generation device according to the first example embodiment;

FIG. 8 is a conceptual diagram illustrating an example of abnormality in gait detected by the gait information generation device according to the first example embodiment;

FIG. 9 is a conceptual diagram for explaining an example of visual information generated by the gait information generation device according to the first example embodiment;

FIG. 10 is a conceptual diagram for describing an arrangement example of a measurement device that measures sensor data acquired by the gait information generation device according to the first example embodiment;

FIG. 11 is a flowchart for explaining an example of the operation of the gait information generation device according to the first example embodiment;

FIG. 12 is a flowchart for explaining an example of a gait information generation process by the gait information generation device according to the first example embodiment;

FIG. 13 is a flowchart for explaining another example of the gait information generation process by the gait information generation device according to the first example embodiment;

FIG. 14 is a conceptual diagram for describing Application Example 1 according to the first example embodiment;

FIG. 15 is a conceptual diagram for describing the Application Example 1 according to the first example embodiment;

FIG. 16 is a conceptual diagram for describing the Application Example 1 according to the first example embodiment;

FIG. 17 is a conceptual diagram for describing Application Example 2 according to the first example embodiment;

FIG. 18 is a conceptual diagram for describing the Application Example 2 according to the first example embodiment;

FIG. 19 is a block diagram illustrating an example of a configuration of a gait information generation device according to a second example embodiment; and

FIG. 20 is a block diagram illustrating an example of a hardware configuration that executes processing according to each example embodiment.

### EXAMPLE EMBODIMENT

Example embodiments of the present invention will be described below with reference to the drawings. In the following example embodiments, technically preferable limitations are imposed to carry out the present invention, but the scope of this invention is not limited to the following description. In all drawings used to describe the following example embodiments, the same reference numerals denote similar parts unless otherwise specified. In addition, in the following example embodiments, a repetitive description of similar configurations or arrangements and operations may be omitted.

### First Example Embodiment

(Configuration)

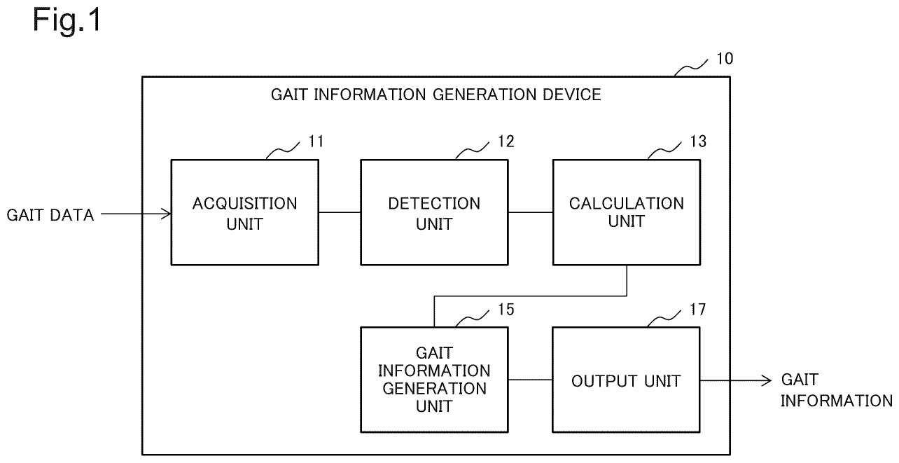

FIG. 1 is a block diagram illustrating a configuration of a gait information generation device 10 according to the present example embodiment. The gait information generation device 10 includes an acquisition unit 11, a detection unit 12, a calculation unit 13, a gait information generation unit 15, and an output unit 17. The acquisition unit 11, the detection unit 12, the calculation unit 13, the gait information generation unit 15, and the output unit 17 may be divided according to the processing to be executed.

The acquisition unit 11 acquires the gait data of the subject. The gait data includes data related to the movement of the foot of the subject. The data related to the movement of the foot is also referred to as foot data. In the present example embodiment, the center position of the foot is referred to as a foot position. The foot position may be shifted from the center position of the foot as long as the verification of the gait movement path is not affected. The foot data is time-series data of a three-dimensional foot position. A method for measuring the foot data is not particularly limited.

For example, foot data is measured by motion capture. In motion capture, a marker is attached to each part of the subject's body. For example, the marker is attached to a site including a foot. A walking subject is photographed with a camera, and a foot position is measured according to a position of a marker in the photographed image (video). According to the motion capture, since the foot position can be directly measured, highly accurate foot data can be obtained.

For example, the foot data is measured by analyzing an image (video) captured by the camera. By using software such as OpenPose, foot data is measured by calculating a foot position based on a position of each of a skeleton and a joint detected from a person in an image.

For example, the foot data is measured using acceleration or angular velocity measured by an inertial sensor attached to the foot. When the inertial sensor is used, the foot position can be calculated by integrating the acceleration and the angular velocity. For example, the foot data may be measured using a smart apparel in which an inertial sensor is attached to each part of the entire body. For example, the foot data is measured according to a foot position measured using an inertial sensor installed on the footwear.

For example, the gait data includes time-series data of foot data in a predetermined gait section. For example, the predetermined gait section includes a plurality of gait cycles. The predetermined gait section may be one gait cycle. In the following description, a period from landing of the heel of the right foot to landing of the heel of the right foot again is defined as one gait cycle of the right foot. Similarly, a period from landing of the heel of the left foot to landing of the heel of the left foot again is defined as one gait cycle of the left foot. The event in which the heel lands is referred to as a heel strike. The heel strike is one of a plurality of events (also referred to as gait events) detected in one gait cycle. The start point and the end point of the gait cycle may be set to the timing of the gait event other than the heel strike.

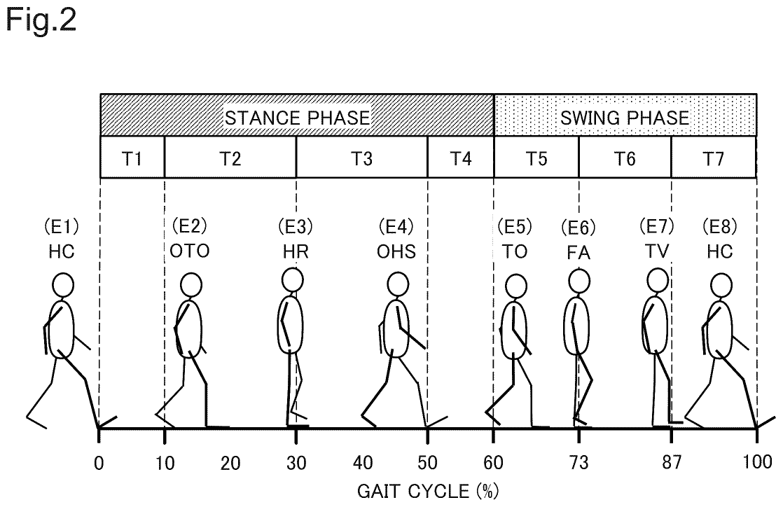

FIG. 2 is a conceptual diagram for explaining a gait event detected in one gait cycle with the right foot as a reference. The horizontal axis of FIG. 2 is a gait cycle normalized with one gait cycle of the right foot as 100% (%). A time point at which the heel of the right foot lands on the ground is defined as a starting point (0%), and a time point at which the heel of the right foot next lands on the ground is defined as an end point (100%). Each of the plurality of timings included in one gait cycle is referred to as a gait phase. The one gait cycle of one foot is roughly divided into a stance phase in which at least part of the back side of the foot is in contact with the ground and a swing phase in which the back side of the foot is away from the ground. In the example of FIG. 2, the gait cycle is normalized in such a way that the stance phase occupies 60% and the swing phase occupies 40%. The stance phase is subdivided into an initial stance period T1, a mid-stance period T2, a terminal stance period T3, and a pre-swing period T4. The swing phase is subdivided into an initial swing period T5, a mid-swing period T6, and a terminal swing period T7. In the gait waveform in one gait cycle, the time point when the heel lands on the ground may not be set as the starting point. For example, the starting point of the gait waveform in one gait cycle may be set as a central time point of the stance phase or the like.

A gait event E1 represents a heel contact (HC), which is the beginning of one gait cycle. The heel strike is an event in which the heel of the right foot, which has been away from the ground in the swing phase, lands on the ground. A gait event E2 represents an opposite toe off (OTO). The opposite toe off is an event in which the toe of the left foot is away from the ground in a state where the ground contact surface of the sole of the right foot is in contact with the ground. A gait event E3 represents a heel rise (HR). The heel rise is an event in which the heel of the right foot is lifted in a state where the ground contact surface of the sole of the right foot is in contact with the ground. A gait event E4 represents an opposite heel strike (OHS). The opposite heel strike is an event in which the heel of the left foot, which has been away from the ground in the swing phase of the left foot, lands on the ground. A gait event E5 represents toe off (TO). The toe off is an event in which the toe of the right foot is away from the ground in a state where the ground contact surface of the sole of the left foot is in contact with the ground. A gait event E6 represents a foot adjacent (FA). The foot adjacent is an event in which the left foot and the right foot cross each other in a state where the ground contact surface of the sole of the left foot is grounded. A gait event E7 represents a tibia vertical (TV). The tibia vertical is an event in which the tibia of the right foot is substantially perpendicular to the ground in a state where the sole of the left foot is grounded. A gait event E8 represents a heel strike (HS) at the end of one gait cycle. A gait event E8 corresponds to the end point of the gait cycle starting from the gait event E1 and corresponds to the starting point of the next gait cycle.

The detection unit 12 extracts foot data from the gait data. The foot data is time-series data of a spatial foot position. The detection unit 12 detects the start point and the end point of the gait cycle from the foot data. In the present example embodiment, the detection unit 12 sets consecutive points of time of heel strike as a start point and an end point for each gait cycle. The heel strike corresponding to the start point is referred to as a first heel strike. The point of the first heel strike is referred to as a first landing point. The heel strike corresponding to the end point is referred to as a second heel strike. The point of the second heel strike is referred to as a second landing point. The foot data includes a spatial foot position for one gait cycle with the first heel strike as a start point and the second heel strike as an end point. For example, with respect to a predetermined gait cycle, the foot data includes a foot position in the horizontal plane at the time point of the first heel strike and a foot position in the horizontal plane at the time point of the second heel strike. The horizontal plane is a plane that horizontally divides the body. When the ground is not inclined, the ground surface is parallel to the horizontal plane.

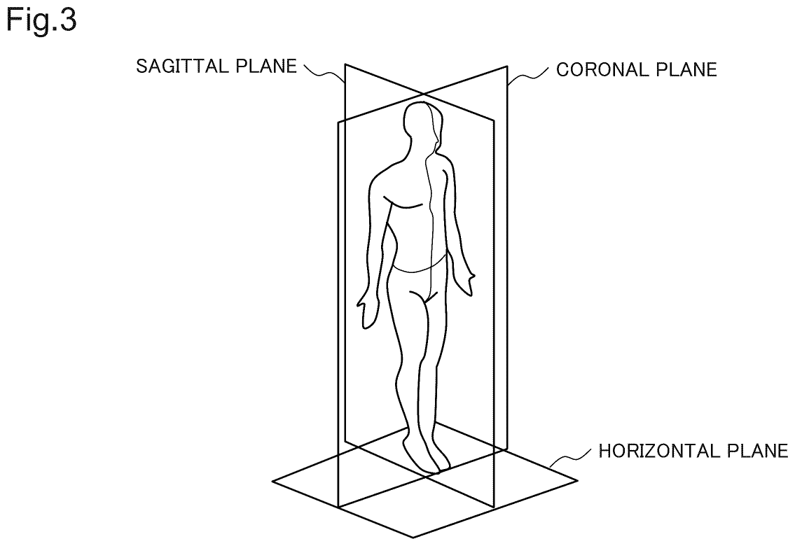

FIG. 3 is a conceptual diagram for describing a face (also referred to as a human body surface) set for the human body. In the present example embodiment, a sagittal plane dividing the body into left and right, a coronal plane dividing the body into front and rear, and a horizontal plane dividing the body horizontally are defined. In the present example embodiment, the ground surface is defined as the horizontal plane. In the present example embodiment, rotation in the sagittal plane with the x-axis as a rotation axis is defined as roll, rotation in the coronal plane with the y-axis as a rotation axis is defined as pitch, and rotation in the horizontal plane with the z-axis as a rotation axis is defined as yaw. A rotation angle in a sagittal plane with the x-axis as a rotation axis is defined as a roll angle, a rotation angle in a coronal plane with the y-axis as a rotation axis is defined as a pitch angle, and a rotation angle in a horizontal plane with the z-axis as a rotation axis is defined as a yaw angle. In the present example embodiment, in the coronal plane, the right side is defined as positive for the right foot, and the left side is defined as positive for the left foot.

The calculation unit 13 calculates a foot movement amount component (also referred to as a first movement amount component) at the time point (start point) of the first heel strike. The calculation unit 13 calculates a foot movement amount component (also referred to as a second movement amount component) at the time point (end point) of the second heel strike. In the present example embodiment, an example of calculating the movement amount component of the foot in the horizontal plane will be described.

For example, the movement amount component of the foot is a three-dimensional acceleration vector. When the movement amount component of the foot is the acceleration vector, the first movement amount component includes the direction and magnitude of the acceleration at the time point (start point) of the first heel strike. When the movement amount component of the foot is the acceleration vector, the second movement amount component includes the direction and magnitude of the acceleration at the time point (start point) of the second heel strike.

For example, the movement amount component of the foot is a three-dimensional velocity vector. When the movement amount component of the foot is the velocity vector, the first movement amount component includes the direction and the magnitude of the velocity at the time point (start point) of the first heel strike. When the movement amount component of the foot is the velocity vector, the second movement amount component includes the direction and magnitude of the velocity at the time point (start point) of the second heel strike.

The gait information generation unit 15 acquires the first movement amount component and the second movement amount component. The gait information generation unit 15 calculates a first auxiliary straight line that passes through the first landing point and is parallel to the first movement amount component. The gait information generation unit 15 calculates a second auxiliary straight line that passes through the second landing point and is parallel to the second movement amount component. The gait information generation unit 15 calculates the gait movement path according to the relationship between the first auxiliary straight line and the second auxiliary straight line. For example, the gait information generation unit 15 calculates the position of the intersection of the first auxiliary straight line and the second auxiliary straight line. The gait information generation unit 15 calculates the gait movement path according to the position of the intersection between the first auxiliary straight line and the second auxiliary straight line.

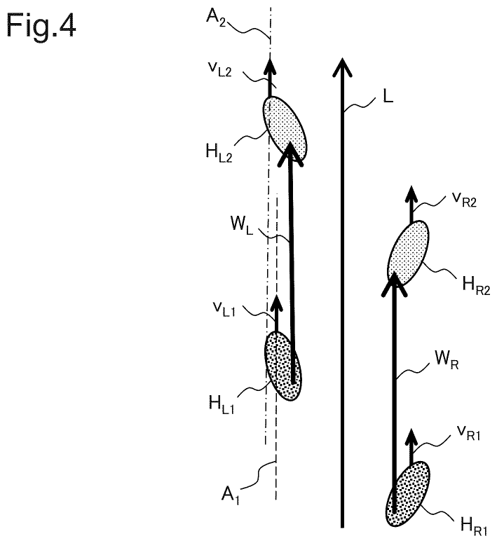

FIG. 4 is a conceptual diagram for explaining a gait movement path W in a case where the gait movement path is a straight line. FIG. 4 illustrates a gait movement path W_{L} related to the left foot and a gait movement path W_{R} related to the right foot. FIG. 4 illustrates a gait path L of the subject. Regarding the gait movement path W_{L} related to the left foot, a first auxiliary straight line A₁ and a second auxiliary straight line A₂ used for deriving the gait movement path W_{L} are illustrated. A first movement amount component v_{L1} is indicated by an arrow (vector) for a first grounding point H_{L1} at the first heel strike of the left foot. The first auxiliary straight line A₁ (broken line) is associated with the first movement amount component v_{L1}. A second movement amount component v_{L2} is indicated by an arrow (vector) for a second grounding point H_{L2} at the second heel strike of the left foot. The second auxiliary straight line A₂ (one-dot chain line) is associated with the second movement amount component v_{L2}.

As illustrated in FIG. 4, when the gait movement path is a straight line, the first auxiliary straight line A₁ (broken line) and the second auxiliary straight line A₂ (one-dot chain line) are substantially parallel. In such a case, the gait information generation unit derives a straight line connecting the first grounding point H_{L1} and the second grounding point H_{L2} as the gait movement path W_{L}. As in the left foot, for the right foot, the gait information generation unit 15 derives the gait movement path W_{R}. FIG. 4 illustrates a first grounding point H_{R1}, a second grounding point H_{R2}, a first movement amount component v_{R1}, and a second movement amount component v_{R2} regarding the right foot. Regarding the right foot, the auxiliary straight line used for deriving the gait movement path W_{R} is omitted.

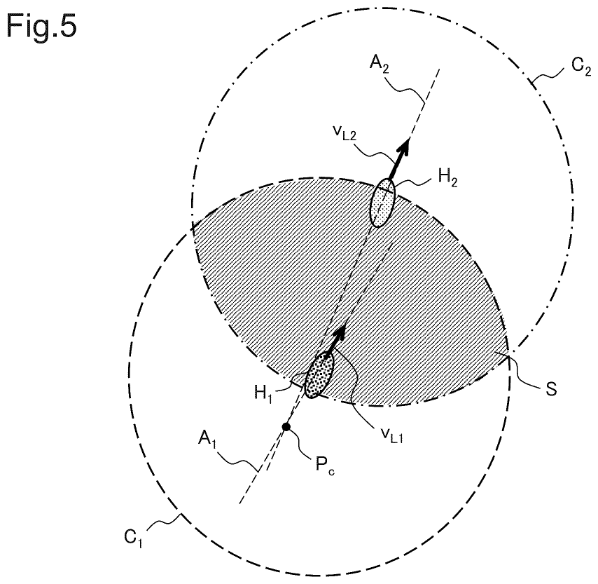

FIG. 5 is a conceptual diagram for describing an example of determining that a gait movement path is a straight line. A region where the first circle C₁ centered around the first grounding point H_{L1} and the second circle C₂ centered around the second grounding point H_{L2} overlap with each other is a specific region S. When the gait movement path is a straight line, an intersection P_{c} between the first auxiliary straight line A₁ (broken line) and the second auxiliary straight line A₂ (one-dot chain line) is located outside the specific region S. That is, when the intersection P_{c} between the first auxiliary straight line A₁ (broken line) and the second auxiliary straight line A₂ (one-dot chain line) is located outside the specific region S, the gait movement path is a straight line.

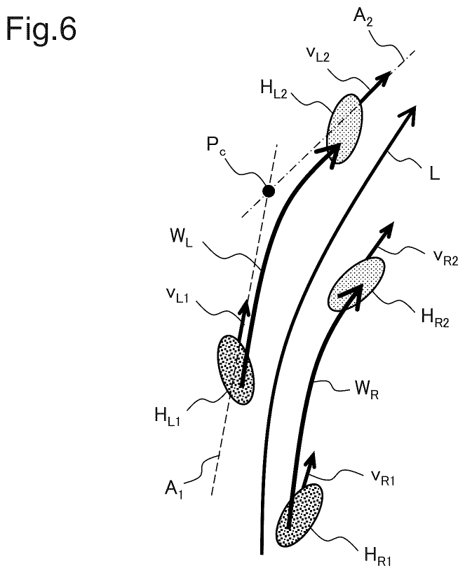

FIG. 6 is a conceptual diagram for explaining the gait movement path W in a case where the gait movement path is a curve. FIG. 6 illustrates the gait movement path W_{L} related to the left foot and the gait movement path W_{R} related to the right foot. FIG. 6 illustrates a gait path L of the subject. Regarding the gait movement path W_{L} related to the left foot, a first auxiliary straight line A₁ and a second auxiliary straight line A₂ used for deriving the gait movement path W_{L} are illustrated. A first movement amount component v_{L1} is indicated by an arrow (vector) for a first grounding point H_{L1} at the first heel strike of the left foot. The first auxiliary straight line A₁ (broken line) is associated with the first movement amount component v_{L1}. A second movement amount component v_{L2} is indicated by an arrow (vector) for a second grounding point H_{L2} at the second heel strike of the left foot. The second auxiliary straight line A₂ (one-dot chain line) is associated with the second movement amount component v_{L2}.

As illustrated in FIG. 6, when the gait movement path is a curved line, the first auxiliary straight line A₁ (broken line) and the second auxiliary straight line A₂ (one-dot chain line) are not parallel. In such a case, the gait information generation unit 15 derives a curve passing through the first grounding point H_{L1}, the second grounding point H_{L2}, and the intersection as the gait movement path. For example, the gait information generation unit 15 derives a curve having the first grounding point H_{L1}, the second grounding point H_{L2}, and the intersection as control points as the gait movement path. For example, the gait information generation unit 15 derives a Bezier curve or a spline curve as a curve with the first grounding point H_{L1}, the second grounding point H_{L2}, and the intersection as control points. In the present example embodiment, an example in which a Bezier curve having the first grounding point H_{L1}, the second grounding point H_{L2}, and the intersection as control points is derived as the gait movement path will be described. As in the left foot, for the right foot, the gait information generation unit 15 derives the gait movement path W_{R}. FIG. 6 illustrates the first grounding point H_{L1}, the second grounding point H_{R2}, the first movement amount component v_{R1}, and the second movement amount component v_{R2} regarding the right foot. Regarding the right foot, the auxiliary straight line used for deriving the gait movement path W_{R} is omitted.

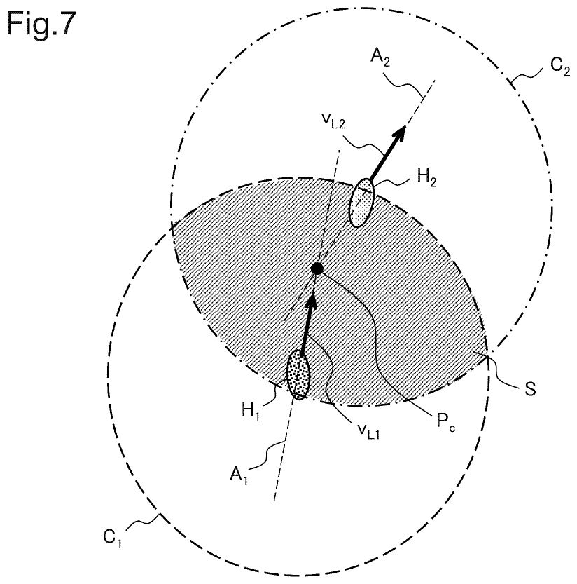

FIG. 7 is a conceptual diagram for describing an example of determining that a gait movement path is a curve. A region where the first circle C₁ centered around the first grounding point H_{L1} and the second circle C₂ centered around the second grounding point H_{L2} overlap with each other is a specific region S. When the gait movement path is a curve, the intersection P_{c} between the first auxiliary straight line A₁ (broken line) and the second auxiliary straight line A₂ (one-dot chain line) is located inside the specific region S. That is, when the intersection P_{c} between the first auxiliary straight line A₁ (broken line) and the second auxiliary straight line A₂ (one-dot chain line) is located inside the specific region S, the gait movement path is a curve.

As described above, when the gait movement path is determined according to the specific region S, the gait information generation unit 15 calculates the position of the intersection P_{c} between the first auxiliary straight line and the second auxiliary straight line. When the intersection P_{c} between the first auxiliary straight line A₁ (broken line) and the second auxiliary straight line A₂ (one-dot chain line) is located outside the specific region S, the gait information generation unit 15 derives a straight line connecting the first grounding point H_{L1} and the second grounding point H_{L2} as the gait movement path W. When the intersection P_{c} of the first auxiliary straight line A₁ (broken line) and the second auxiliary straight line A₂ (one-dot chain line) is located inside the specific region S, the gait information generation unit 15 derives a Bezier curve having the first grounding point H_{L1}, the second grounding point H_{L2}, and the intersection P_{c} as control points as the gait movement path.

For example, the gait information generation unit 15 may derive a Bezier curve having the first grounding point H_{L1}, the second grounding point H_{L2}, and the intersection as control points as the gait movement path regardless of the position of the intersection between the first auxiliary straight line and the second auxiliary straight line. In this case, when the gait movement path is a straight line, it is not possible to derive an accurate gait movement path. Therefore, as described above, it is better to switch the method of deriving the gait movement path according to the position of the intersection between the first auxiliary straight line and the second auxiliary straight line.

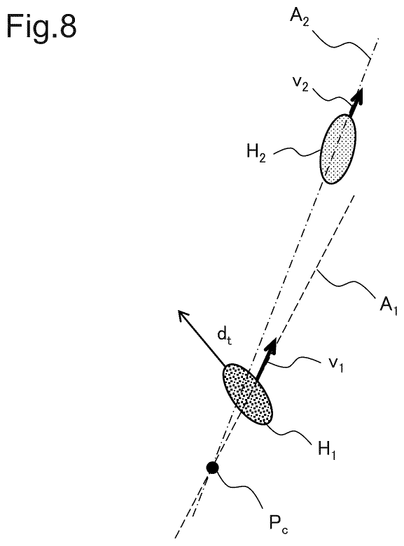

FIG. 8 is a conceptual diagram for describing determination of a gait movement path in a case where an abnormality occurs during gait. A first movement amount component v_{L1} is indicated by an arrow (vector) for a first grounding point H_{L1} at the first heel strike of the left foot. The first auxiliary straight line A₁ (broken line) is associated with the first movement amount component v_{L1}. A second movement amount component v_{L2} is indicated by an arrow (vector) for a second grounding point H_{L2} at the second heel strike of the left foot. The second auxiliary straight line A₂ (one-dot chain line) is associated with the second movement amount component v_{L2}. In the case of the example of FIG. 8, a direction of the first movement amount component v_{L1} and a direction dₜ of the toe greatly deviate at the first grounding point H_{L1}. In such a case, it can be estimated that some abnormality has occurred.

For example, as the abnormality, an event of twisting the ankle, stumbling over a step, losing balance, or falling down is assumed. In such an event, the deviation between the direction of the first movement amount component v_{L1} and the direction dₜ of the toe is larger than usual. Therefore, the abnormality in gait may be detected according to the deviation between the direction of the first movement amount component v_{L1} and the direction dₜ of the toe. For example, the gait information generation unit 15 detects the occurrence of abnormality in a case where the direction of the first movement amount component v_{L1} and the direction dₜ of the toe deviate by a preset reference angle or more. Similarly, the gait information generation unit 15 detects the occurrence of abnormality according to the deviation between a direction of the second movement amount component v_{L2} and a direction dₜ of the toe. The gait information generation unit 15 may detect the occurrence of the abnormality according to the variation in the deviation between the direction of each of the first movement amount component vu and the second movement amount component v_{L2} and the direction dₜ of the toe in a plurality of consecutive gait cycles. Detection of occurrence of abnormality is not particularly limited.

For example, there is a possibility that some kind of abnormality has occurred in a subject who is walking on a straight road and whose gait movement path swing left and right. For example, when the subject is drunk and staggered, the gait movement path continues to swing left and right. In such a case, when a notification is given to a family member of the subject, the family member can go to pick up the subject.

The gait information generation unit 15 generates gait information about the derived gait movement path. For example, the gait information includes visual information about a gait movement path according to gait of the subject. For example, the visual information about the gait movement path is a curved line or a straight line connecting the first grounding point H_{L1} and the second grounding point H_{L2}. For example, the visual information about the gait movement path is an arrow having the first grounding point H_{L1} as a start point and the second grounding point H_{L2} as an end point. For example, the visual information about the gait movement path is superimposed on the video indicating the gait of the subject. The gait information is not particularly limited as long as it includes visual information about a gait movement path.

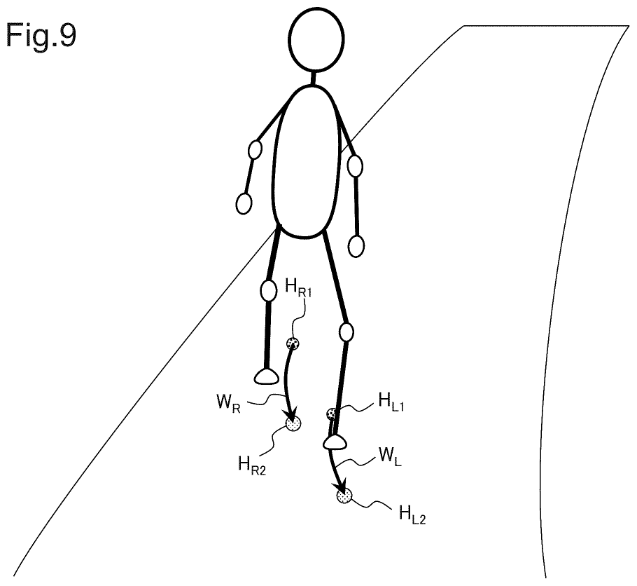

FIG. 9 is a conceptual diagram for explaining an example of visual information about a gait movement path. FIG. 9 is a conceptual diagram of a walking person (character) viewed from the front upper side. The person in FIG. 9 is walking on a road turning to the left with the person at the center. The person in FIG. 9 has the left foot as a support leg and is in a state in which the right foot is away from the ground. FIG. 9 illustrates the first grounding point H_{L1}, the second grounding point H_{L2}, and the gait movement path W_{L} with respect to the left foot. FIG. 9 illustrates a first grounding point H_{R1}, a second grounding point H_{R2}, and a gait movement path W_{R} with respect to the right foot. In the example of FIG. 9, the curved line indicating the gait movement path W includes an arrowhead indicating the traveling direction. The curved line indicating the gait movement path W may not include the arrowhead indicating the traveling direction. The gait movement path W may be expressed by one curved line obtained by averaging the gait movement path W_{R} regarding the right foot and the gait movement path W_{L} regarding the left foot. The viewpoint for a walking person can be set in any manner in a left-right direction, a rear direction, an upper direction, an oblique direction, or the like with the person at the center. For example, not the entire body of the person but only a portion below the waist (lower body) may be displayed on the video. The gait movement path W is changed in accordance with the gait of the person in the video. For example, the gait movement path W is changed in association with the gait cycle of the person in the video. According to the example of FIG. 9, it is possible to intuitively grasp the gait state including the change in the traveling direction according to the movement of the gait movement path W that varies according to the gait of the person in the video.

The output unit 17 outputs the gait information generated by the gait information generation unit 15. For example, the output unit 17 outputs gait information to a terminal device having a screen. The gait information output to the terminal device is displayed on the screen of the terminal device. For example, the output unit 17 displays gait information on a screen of a mobile terminal of the subject (user). For example, the output unit 17 displays gait information on a screen of a terminal device used by an expert such as a medical doctor, a physical therapist, or a caregiver who verifies the physical condition of the subject. The expert can give a diagnosis or advice according to the gait information displayed on the screen of the terminal device to the subject. For example, the output unit 17 may output the gait information to an external system or the like that uses the gait information. The use of the gait information output from the output unit 17 is not particularly limited.

For example, the gait information generation device 10 is connected to an external system or the like constructed in a cloud or a server via a mobile terminal (not illustrated) carried by a subject (user). The mobile terminal (not illustrated) is a portable communication device. For example, the mobile terminal is a portable terminal device having a communication function, such as a smartphone, a smart watch, a tablet, or a mobile telephone.

For example, the gait information generation device 10 is connected to a terminal device (not illustrated) used by a person who verifies the physical condition of the subject (user). Software for processing gait information and displaying an image related to the gait information is installed in the terminal device. For example, the terminal device is an information processing device such as a stationary personal computer, a notebook personal computer, a tablet, or a mobile terminal. The terminal device may be a dedicated terminal that processes the gait information.

For example, the gait information generation device 10 is connected to a mobile terminal or a terminal device via a wire such as a cable. For example, the gait information generation device 10 is connected to a mobile terminal or a terminal device via wireless communication. For example, the gait information generation device 10 is connected to a mobile terminal or a terminal device via a wireless communication function (not illustrated) conforming to a standard such as Bluetooth (registered trademark) or WiFi (registered trademark). The communication function of the gait information generation device 10 may conform to a standard other than Bluetooth (registered trademark) or WiFi (registered trademark). The gait information may be used by an application installed in a mobile terminal or a terminal device. In this case, the mobile terminal or the terminal device executes processing using the gait information by application software or the like installed in the device. The gait information generation device 10 may be mounted on a mobile terminal or a terminal device.

### [Measurement Device]

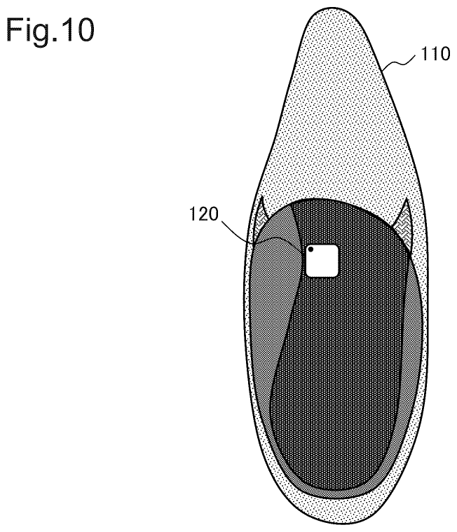

Next, an example of a measurement device that measures foot data will be described with reference to the drawings. FIG. 10 is a conceptual diagram illustrating an example in which a measurement device 120 including a sensor that measures a physical quantity related to the movement of the foot is disposed in a shoe 110. In the present example embodiment, an example in which the measurement device 120 is disposed in the shoe 110 will be described. The measurement device 120 may be attached to the waist, the knee, or the like as long as it can measure a physical quantity related to the movement of the foot.

FIG. 10 illustrates an example in which the measurement device 120 is disposed in the shoe 110 of one foot (right foot). In order to derive the gait movement paths of both feet, the measurement device 120 may be disposed on each of the shoes 110 of both feet. In the example of FIG. 10, the measurement device 120 is installed at a position corresponding to the back side of the arch of foot. For example, the measurement device 120 is mounted on an insole inserted into the shoe 110. For example, the measurement device 120 may be mounted on the bottom face of the shoe 110. For example, the measurement device 120 may be embedded in the main body of the shoe 110. The measurement device 120 may be detachable from the shoe 110 or may not be detachable from the shoe 110. The measurement device 120 may be installed at a position other than the back side of the arch of the foot as long as the sensor data regarding the movement of the foot can be acquired. The measurement device 120 may be installed on a sock worn by the user or a decorative article such as an anklet worn by the user. The measurement device 120 may be directly attached to the foot or embedded in the foot.

The measurement device 120 includes an acceleration sensor and an angular velocity sensor. The acceleration sensor is a sensor that measures accelerations in three axial directions (also referred to as spatial accelerations). The acceleration sensor measures accelerations in three axial directions (also referred to as spatial accelerations) as physical quantities related to the movement of the foot. The acceleration sensor outputs the measured spatial acceleration. The angular velocity sensor is a sensor that measures angular velocities around three axes (also referred to as spatial angular velocities). The angular velocity sensor measures angular velocities around three axes (also referred to as spatial angular velocities) as physical quantities related to the movement of the foot. The angular velocity sensor outputs the measured spatial angular velocity. The measurement device 120 stores sensor data related to the measured physical quantity in a buffer (not illustrated). The data format of the sensor data is not particularly limited.

For example, a piezoelectric sensor, a piezoresistive sensor, or a capacitance type sensor can be used as the acceleration sensor. The sensor used as the acceleration sensor does not have the limited measurement method as long as the sensor can measure the acceleration. For example, a vibration type sensor, a capacitance type sensor, or the like can be used as the angular velocity sensor. The sensor used as the angular velocity sensor have the limited measurement method as long as the sensor can measure the angular velocity. The measurement device 120 may include a sensor other than the acceleration sensor and the angular velocity sensor. The description of the sensor other than the acceleration sensor and the angular velocity sensor that can be included in the measurement device 120 is omitted.

The measurement device 120 is, for example, an inertial measurement device that measures acceleration or angular velocity. An example of the inertial measurement device is an inertial measurement unit (IMU). The IMU includes an acceleration sensor that measures acceleration in three axial directions and an angular velocity sensor that measures angular velocities around the three axes. The measurement device 120 may be achieved by an inertial measurement device such as a vertical gyro (VG) or an attitude heading reference system (AHRS). The measurement device 120 may be achieved by a global positioning system/inertial navigation system (GPS/INS).

The measurement device 120 transmits the sensor data stored in the buffer at a prescribed timing. For example, the measurement device 120 transmits the gait parameter during the swing phase in which the measurement of the sensor data is hardly affected. For example, the measurement device 120 may transmit sensor data for each gait cycle. For example, the measurement device 120 may transmit sensor data every predetermined time period. The measurement device 120 deletes the transmitted sensor data used to calculate the gait parameter from the buffer. The sensor data transmitted from the measurement device 120 may be all data for one gait cycle or data in a specific time zone. The sensor data transmitted from the measurement device 120 may be converted into the world coordinate system or may remain in the local coordinate system. The sensor data transmitted from the measurement device 120 may be a specific gait parameter. For example, the specific gait parameter includes a gait velocity, a stride length, a grounding angle, a ground leaving angle, a foot raising height (sensor position), an outward rotation angle, a direction of a toe, and the like. The specific gait parameter may not include all of the above-described gait parameters, or may include other gait parameters.

For example, the sensor data transmitted from the measurement device 120 is received by a mobile terminal (not illustrated) carried by the user wearing the shoe 110 on which the measurement device 120 is disposed. The measurement device 120 may transmit sensor data via a wire such as a cable or may transmit sensor data via wireless communication. For example, the measurement device 120 is configured to transmit sensor data via a wireless communication function (not illustrated) conforming to a standard such as Bluetooth (registered trademark). The communication function of the measurement device 120 may conform to a standard other than Bluetooth (registered trademark).

The mobile terminal (not illustrated) receives the sensor data transmitted from the measurement device 120. For example, the mobile terminal executes a process of generating gait information using the received sensor data by application software or the like installed in the mobile terminal. For example, the mobile terminal displays the generated gait information on a screen of the mobile terminal. For example, the generated gait information may be displayed on a screen of a terminal device (not illustrated) visible by the user. The mobile terminal may transmit the received sensor data to a server, a cloud, or the like. The use of the sensor data received by the mobile terminal is not particularly limited.

(Operation)

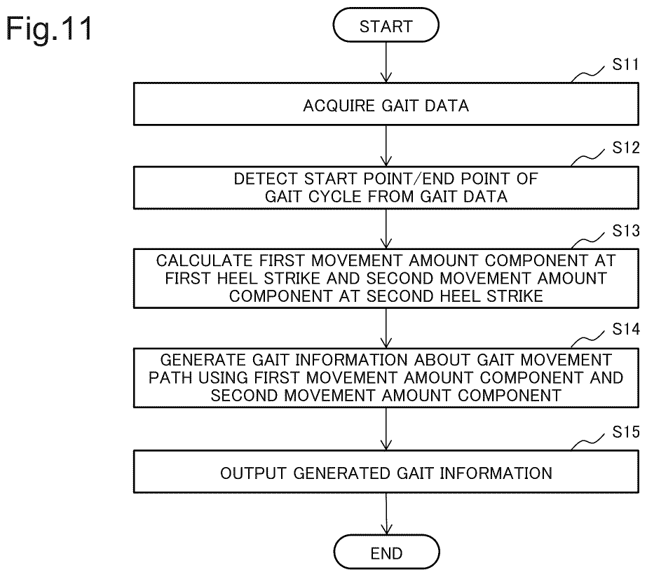

Next, an example of the operation of the gait information generation device 10 will be described with reference to the drawings. FIG. 11 is a flowchart for explaining an example of the operation of the gait information generation device 10. In the description along the flowchart of FIG. 11, the gait information generation device 10 is used as an operation subject.

In FIG. 11, first, the gait information generation device 10 acquires gait data (step S11). The gait data includes time-series data (foot data) of the foot position according to the physical quantity related to the movement of the foot.

Next, the gait information generation device 10 detects the start point and the end point of the gait cycle from the foot data included in the gait data (step S12). For example, the gait information generation device 10 detects consecutive heel strikes as the start point and the end point of the foot data. Of the consecutive heel strikes, the preceding heel strike point is the start point. Of the consecutive heel strikes, the subsequent heel strike point is the end point.

Next, the gait information generation device 10 calculates a first movement amount component at the time point (start point) of the first heel strike and a second movement amount component at the time point (end point) of the second heel strike (step S13). The first movement amount component is an acceleration vector or a velocity vector at the time point (start point) of the first heel strike. The second movement amount component is an acceleration vector or a velocity vector at the time point (end point) of the second heel strike.

Next, the gait information generation device 10 generates gait information about the gait movement path using the first movement amount component and the second movement amount component (step S14). The gait information includes visual information indicating a gait movement path.

Next, the gait information generation device 10 outputs the generated gait information (step S15). The gait information generation device 10 outputs gait information including visual information about a gait movement path. The visual information included in the output gait information is displayed on a screen of a mobile terminal, a terminal device (not illustrated), or the like.

### [Gait Information Generation Process]

Next, an example of step S14 (gait information generation process) in FIG. 11 will be described with reference to the drawings. Hereinafter, an example in which the same processing is performed regardless of whether the gait movement path is a straight line or a curved line, and an example in which different processing is performed between a case where the gait movement path is a straight line and a case where it is a curved line will be described. In the following gait information generation process, the gait information generation device 10 is set an operation subject.

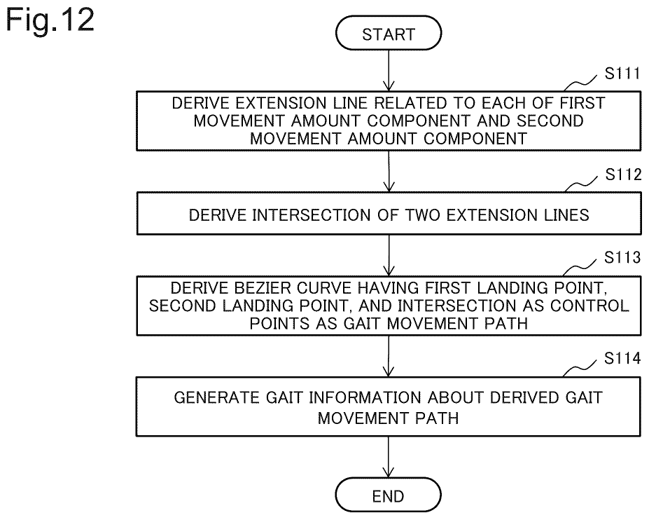

FIG. 12 is a flowchart for explaining an example of the gait information generation process. FIG. 12 is an example in which the same processing is performed regardless of whether the gait movement path is a straight line or a curved line.

In FIG. 12, first, the gait information generation device 10 derives an extension line regarding each of the first movement amount component and the second movement amount component (step S111).

Next, the gait information generation device 10 derives an intersection of two extension lines (step S112).

Next, the gait information generation device 10 derives a Bezier curve having the first landing point, the second landing point, and the intersection as control points as the gait movement path (step S113).

Next, the gait information generation device 10 generates gait information about the derived gait movement path (step S114).

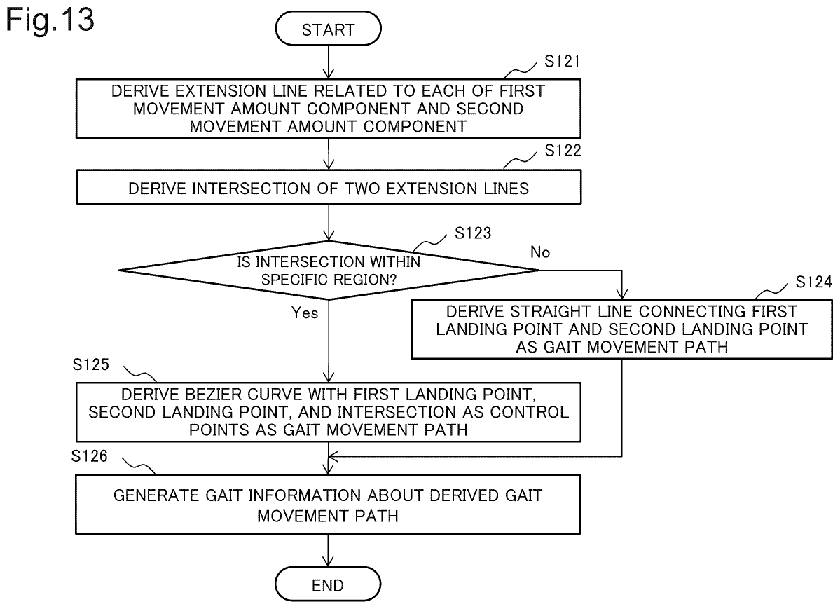

FIG. 13 is a flowchart for explaining another example of the gait information generation process. FIG. 13 illustrates an example in which different processing is performed between a case where the gait movement path is a straight line and a case where it is a curved line.

In FIG. 13, first, the gait information generation device 10 derives an extension line regarding each of the first movement amount component and the second movement amount component (step S121).

Next, the gait information generation device 10 derives an intersection of two extension lines (step S122).

When the position of the intersection is not within the range of the specific region (No in step S123), the gait information generation device 10 derives a straight line connecting the first landing point and the second landing point as the gait movement path (step S124). After step S123, the process proceeds to step S126.

On the other hand, when the intersection is within the range of the specific region (Yes in step S123), the gait information generation device 10 derives a Bezier curve having the first landing point, the second landing point, and the intersection as control points as the gait movement path (step S125).

After steps S123 and S124, the gait information generation device 10 generates gait information about the derived gait movement path (step S126).

In the case of the gait information generation process of FIG. 12, when the intersection of the extension lines regarding each of the first movement amount component and the second movement amount component deviates from the specific region, it is not possible to derive an appropriate gait movement path. In the case of the gait information generation process of FIG. 13, even when the intersection of the extension lines deviates from the specific region, it is not possible to derive an appropriate gait movement path. Therefore, according to the gait information generation process of FIG. 13, even when the gait movement path is a straight line, it is possible to derive an appropriate gait movement path.

### APPLICATION EXAMPLE

Next, an application example regarding the gait information generation device will be described with reference to the drawings. In the following application example, an example will be described in which gait information (also referred to as display-information) including visual information is superimposed on a video of a walking subject when looked down from obliquely above and displayed. The video of the subject may be an actual video or a virtual person (character) imitating the motion of the subject. Hereinafter, an example of displaying a character in a video will be described. The display-information described below may be generated by the gait information generation device 10 or may be generated by another device or system that has acquired the gait information.

### Application Example 1

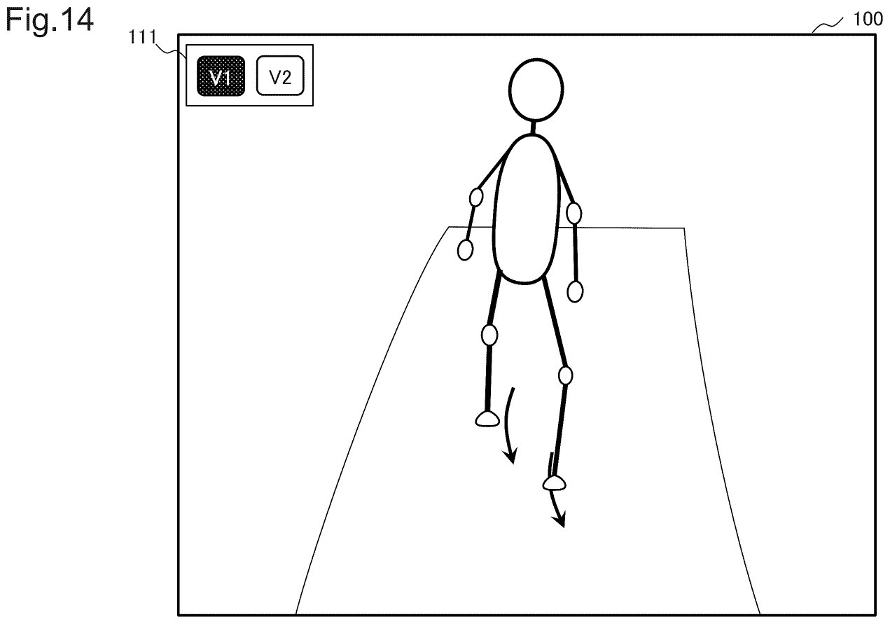

FIGS. 14 to 16 are conceptual diagrams related to Application Example 1 related to the gait information generation device 10. In the present application example, an arrow indicating the gait movement path is displayed on a screen 100. In the present application example, an arrow indicating a gait movement path is displayed with superimposed on the foot of the person (character). The arrow indicating the gait movement path may be displayed at a position away from the person (character).

A viewpoint switching region 111 including a button for switching the viewpoint is displayed at the position of the upper left corner of the screen 100. In the viewpoint switching region 111, buttons for switching the viewpoint between a front viewpoint (first viewpoint V1) and a left obliquely front viewpoint (second viewpoint V2) with the person (character) at the center are displayed. The viewpoint is related to the viewpoint of the user viewing the screen 100. The viewpoint switching region 111 is an interface region that receives a user's operation.

FIG. 14 illustrates an example in which a video of a person (character) when viewed from a front viewpoint (first viewpoint V1) with the person (character) at the center is displayed on the screen 100. In FIG. 14, the first viewpoint V1 is selected in the viewpoint switching region 111. When viewed from the first viewpoint V1, the gait movement path can be grasped while referring to the gait state in the coronal plane. That is, when viewed from the first viewpoint V1, it is easy to grasp the gait state in the left-right direction (in the coronal plane).

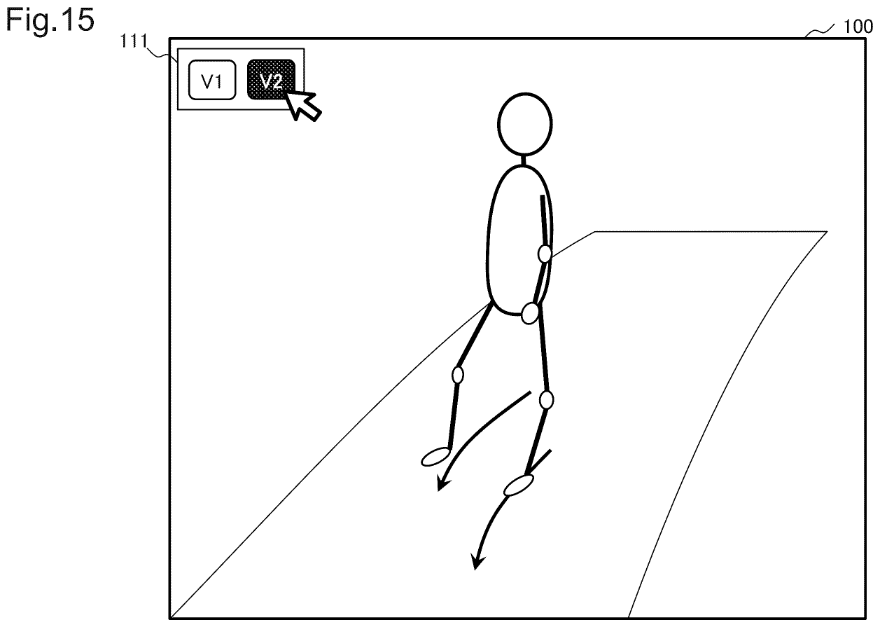

FIG. 15 illustrates an example in which a video of a person (character) when viewed from a left obliquely front viewpoint (second viewpoint V2) with the person (character) at the center is displayed on the screen 100. FIG. 15 illustrates a state in which the viewpoint has been switched from the first viewpoint V1 (FIG. 14) to the second viewpoint V2 in response to the selection of the second viewpoint V2 in the viewpoint switching region 111. When viewed from an obliquely front viewpoint with respect to the traveling direction of the person (character) as in the second viewpoint V2, it is easy to three-dimensionally grasp the change in the traveling direction.

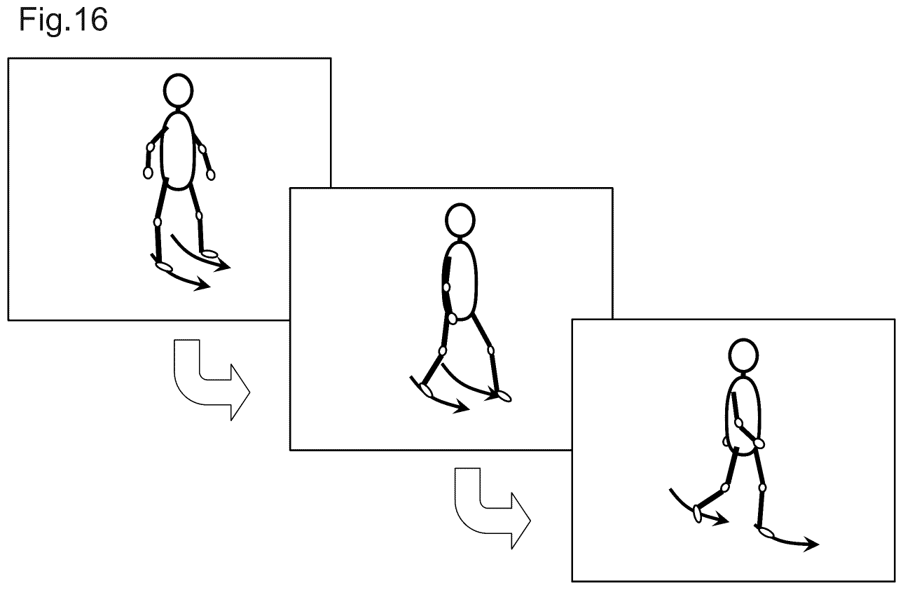

FIG. 16 is a conceptual diagram illustrating an example in which a gait movement path in the present application example is displayed in association with a moving image. FIG. 16 illustrates a state of a person (character) when viewed from a right obliquely front viewpoint with the person (character) at the center. FIG. 16 illustrates three frames extracted from a plurality of frames included in a video related to gait of a person. The actual video is composed of more frames. In the three frames in FIG. 16, time (gait cycle) progresses from the upper left to the lower right. The video indicating the gait state of the person changes according to the progress of time (gait cycle). The arrow indicating the gait movement path changes in accordance with the gait phase in the gait of the person. By superimposing the arrow indicating the gait movement path on the video, it is possible to intuitively grasp the change in the traveling direction of the person according to the gait movement path that varies according to the gait of the person.

### Application Example 2

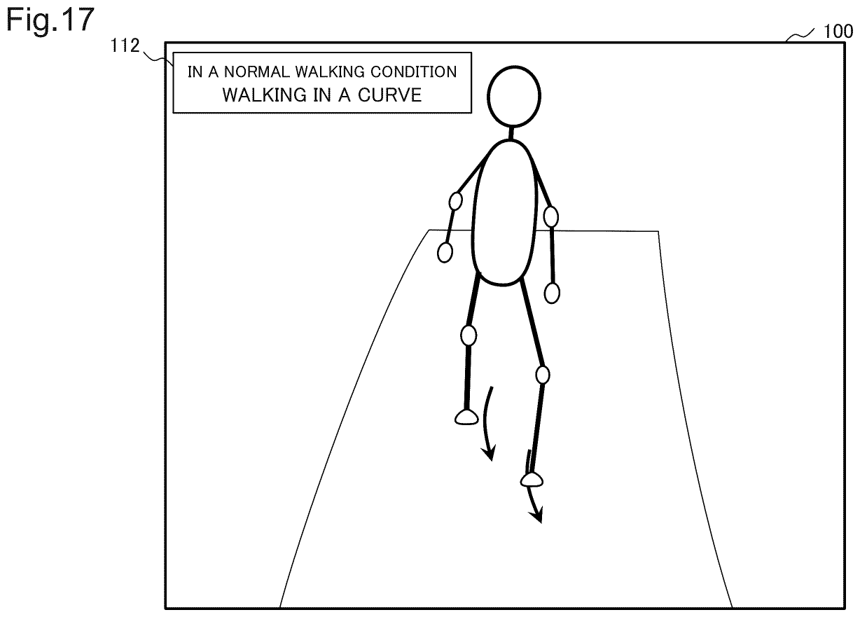

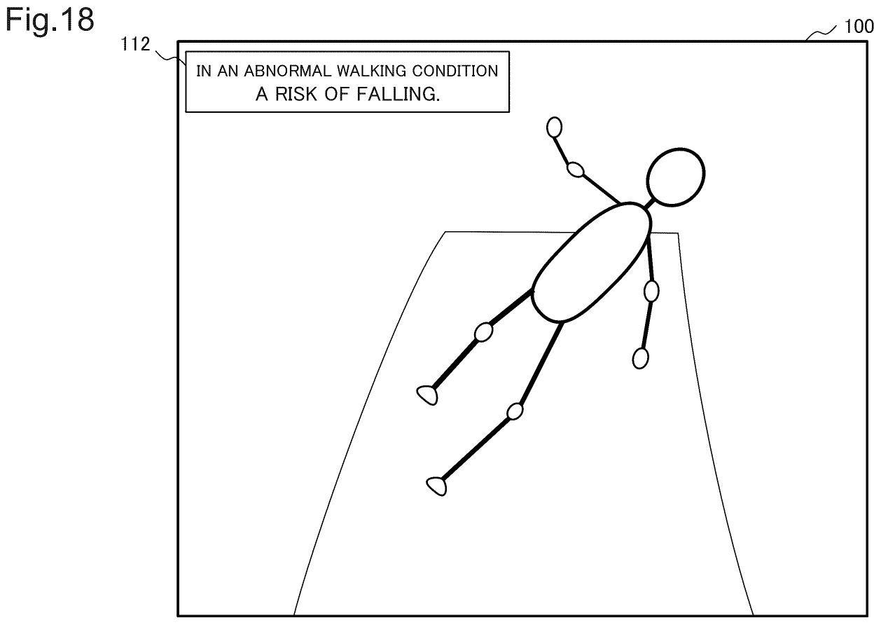

FIGS. 17 to 18 are conceptual diagrams related to Application Example 2 related to the gait information generation device 10. In the present application example, an arrow indicating the gait movement path is displayed on a screen 100. An information display region 112 is displayed at the position of the upper left corner of the screen 100. The information display region 112 is a display region in which information related to the gait movement path is displayed.

FIG. 17 is an example in which an image indicating a situation where a person (character) is walking in a normal gait state is displayed on the screen 100. In the example of FIG. 17, information that “IN A NORMAL WALKING CONDITION” is displayed on the screen 100 in accordance with the gait state of the person who normally walks. In the example of FIG. 17, information that “WALKING IN A CURVE” is displayed on the screen 100 according to the curved gait movement path.

FIG. 18 illustrates an example in which an image indicating a situation in which a person (character) is likely to fall is displayed on the screen 100. In the example of FIG. 18, information that “IN AN ABNORMAL WALKING CONDITION” is displayed on the screen 100 in response to the detection of the abnormality of the gait movement path. In the example of FIG. 18, information that “A RISK OF FALLING” is displayed on the screen 100 according to the detected abnormality. When the abnormality of the subject is detected in real time, a warning may be issued in response to the detection of the abnormality. For example, when a warning sound is issued from a mobile terminal carried by the subject, people surrounding the subject can notice an abnormality of the subject. For example, the occurrence of the abnormality in the subject may be notified to a family member or an acquaintance of the subject. In this way, the family member or the acquaintance who has received the notification can take some action.

In the present application example, information indicating the gait state of the person in the video together with the gait movement path is displayed on the screen 100.

In the present application example, information indicating that an abnormality has occurred in the person in the video is displayed on the screen 100. Therefore, according to the present application example, the state of the person in the video can be intuitively grasped.

As described above, the gait information generation device according to the present example embodiment includes the acquisition unit, the detection unit, the calculation unit, the gait information generation unit, and the output unit. The acquisition unit acquires gait data including time-series data of the foot position of the subject. The detection unit detects the start point and the end point of the gait cycle from the time-series data of the foot position included in the gait data. The calculation unit calculates a first movement amount component related to a start point and a second movement amount component related to an end point. The gait information generation unit generates the gait information about the gait movement path of the subject using the calculated first movement amount component and the calculated second movement amount component, and the output unit outputs the generated gait information.

In the present example embodiment, for each of both feet of the subject, the gait information about the gait movement path according to the movement amount component of each foot is generated. Therefore, according to the present example embodiment, the gait information about the gait movement path for each gait cycle can be generated for each of both feet.

In an aspect of the present example embodiment, the calculation unit calculates the acceleration vector or the velocity vector at the start point as the first movement amount component. The calculation unit calculates the acceleration vector or the velocity vector at the end point as the second movement amount component. According to the present aspect, it is possible to derive the gait movement path for each gait cycle for each of both feet by using the acceleration vector or the velocity vector as the movement amount component.

In an aspect of the present example embodiment, the acquisition unit detects a preceding first heel strike as a start point among two consecutive heel strikes. The acquisition unit detects a subsequent second heel strike as an end point among two consecutive heel strikes. The calculation unit calculates a position of an intersection of a first auxiliary straight line that is parallel to the first movement amount component and passes through the start point and a second auxiliary straight line that is parallel to the second movement amount component and passes through the end point in the horizontal plane. The gait information generation unit derives a curved line passing through the start point, the end point, and the intersection as a gait movement path. According to the present aspect, the curved line passing through the start point, the end point, and the intersection can be derived as the gait movement path for each gait cycle.

In an aspect of the present example embodiment, the gait information generation unit derives a curved line having the start point, the end point, and the intersection as control points as a gait movement path. According to the present aspect, a curved line having the start point, the end point, and the intersection as control points can be derived as a gait movement path for each gait cycle.

In an aspect of the present example embodiment, the gait information generation unit derives a Bezier curved line having the start point, the end point, and the intersection as control points as a gait movement path. According to the present aspect, the Bezier curved line having the start point, the end point, and the intersection as the control points can be derived as the gait movement path for each gait cycle.

In an aspect of the present example embodiment, the gait information generation unit derives the gait movement path according to the positional relationship between the intersection and the specific region where the first circle and the second circle overlap with each other. The first circle is a circle centered around the start point with the distance between the start point and the end point as a radius. The second circle is a circle centered around the end point with the distance between the start point and the end point as a radius. In a case where the intersection is located inside the specific region, the gait information generation unit derives a curved line passing through the start point, the end point, and the intersection as the gait movement path. When the intersection is located outside the specific region, the gait information generation unit derives a straight line connecting the start point and the end point as the gait movement path. According to the present aspect, it is possible to select an appropriate gait movement path according to the positional relationship between the specific region and the intersection.

In an aspect of the present example embodiment, the gait information generation unit generates gait information in which visual information including a gait movement path is superimposed on a frame constituting a video indicating a gait state of the subject. The output unit outputs gait information about the subject to the terminal device. The output unit displays the display-information about the visual information included in the gait information on the screen of the terminal device. According to the present aspect, it is easy to intuitively grasp the gait movement path of each of both feet in accordance with the gait state of the subject displayed on the screen of the terminal device.

In an aspect of the present example embodiment, the gait information generation unit generates gait information including visual information in which information related to the detected abnormality is superimposed on a frame constituting a video indicating a gait state of the subject. The abnormality is detected from at least one of the first movement amount component and the second movement amount component related to the subject. The output unit outputs gait information about the subject to the terminal device. The output unit displays the display-information about the visual information included in the gait information on the screen of the terminal device. According to the present aspect, it is possible to intuitively grasp the abnormality occurring in the subject in accordance with the gait state of the subject displayed on the screen of the terminal device.

The method of the present example embodiment can also be applied to gait on a road surface that is not flat. For example, the method of the present example embodiment can also be applied to gait on a slope or a staircase. According to the method of the present example embodiment, it is possible to evaluate lateral status when the subject is walking on a curve by visual information indicating a gait movement path of the subject. For example, the technique of the present example embodiment may be used to detect that the subject is walking on a curve. For example, when the falling risk is notified to the subject according to the curvature of the curve, there is a possibility that the falling of the subject on the curve might be prevented. For example, the physical condition of the subject may be estimated according to the detection of the curved gait.

There is a possibility that a subject who is walking while swinging from side to side periodically on a straight road is drunk and staggered. In such a case, when a family member or a person around the subject is notified that there is an abnormality in the physical condition of the subject, there is a possibility that the danger that may reach the subject may be avoided by the support of the family member or the person around the subject. At a platform of a station, an accident may occur in which a person who is drunk falls onto a railway track. When the abnormality of the physical condition of the subject detected by the method of the present example embodiment is notified to a station staff or a passenger around the subject, there is a possibility that a fall accident to the track may be reduced.

In a case where circumduction of the subject is large, the gait movement path is curved even if the subject walks straight. In this case, the curvature centers of the gait movement path derived with respect to both feet are located on the opposite sides across the movement line of the center of gravity of the subject. In such a case, the gait movement path derived for each of both feet corresponds to the locus of the circumduction of each of the both feet. In one gait cycle, the circumduction amount is maximized at the time point when the length of the perpendicular drawn down from the gait movement path to the straight line indicating the movement of the center of gravity of the body is maximized. For example, a path obtained by averaging the gait movement paths derived for both feet can be regarded as a straight line indicating the center-of-gravity movement of the body. Therefore, the gait movement path in the horizontal plane that can be derived by the method of the present example embodiment can be used as an index of circumduction.

### Second Example Embodiment

Next, a gait information generation device according to the second example embodiment will be described with reference to the drawings. The gait information generation device of the present example embodiment has a configuration in which the first gait information generation device is simplified.

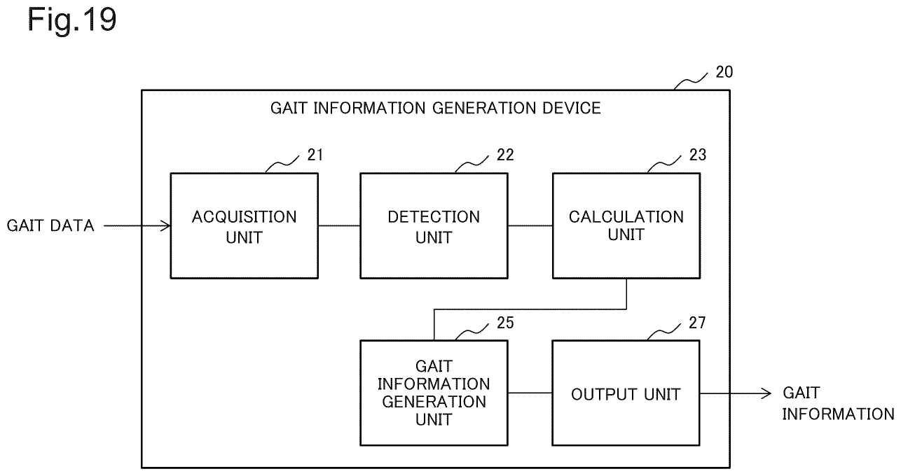

FIG. 19 is a block diagram illustrating an example of a configuration of a gait information generation device 20 according to the present example embodiment. The gait information generation device 20 includes an acquisition unit 21, a detection unit 22, a calculation unit 23, a gait information generation unit 25, and an output unit 27.

The acquisition unit 21 acquires gait data including time-series data of the foot position of the subject. The detection unit 22 detects the start point and the end point of the gait cycle from the time-series data of the foot position included in the gait data. The calculation unit 23 calculates a first movement amount component related to the start point and a second movement amount component related to the end point. The gait information generation unit 25 generates the gait information about the gait movement path of the subject using the calculated first movement amount component and the calculated second movement amount component. The output unit 27 outputs the generated gait information.

In the present example embodiment, for each of both feet of the subject, the gait information about the gait movement path according to the movement amount component of each foot is generated. Therefore, according to the present example embodiment, the gait information about the gait movement path for each gait cycle can be generated for each of both feet.

(Hardware)

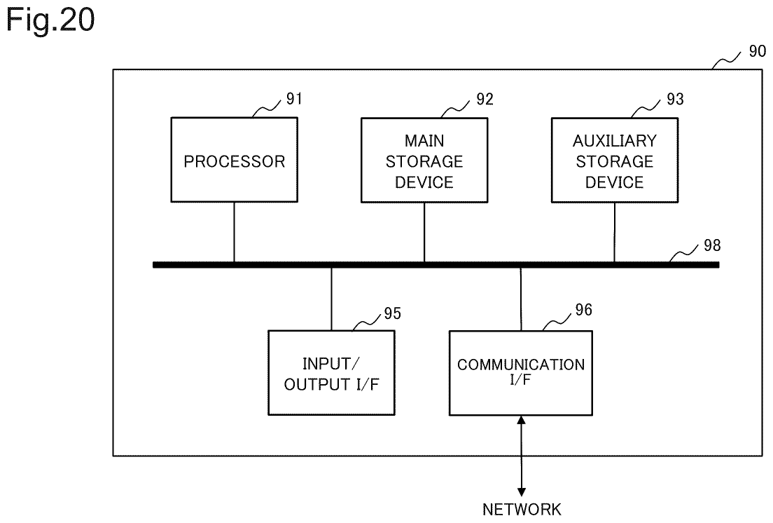

A hardware configuration for executing the processing according to each example embodiment of the present disclosure will be described using an information processing device 90 (computer) of FIG. 20 as an example. The information processing device 90 in FIG. 20 is a configuration example for executing the processing of each example embodiment, and does not limit the scope of the present disclosure.

As illustrated in FIG. 20, the information processing device 90 includes a processor 91, a main storage device 92, an auxiliary storage device 93, an input/output interface 95, and a communication interface 96. In FIG. 20, the interface is abbreviated as an interface (I/F). The processor 91, the main storage device 92, the auxiliary storage device 93, the input/output interface 95, and the communication interface 96 are data-communicably connected to each other via a bus 98. The processor 91, the main storage device 92, the auxiliary storage device 93, and the input/output interface 95 are connected to a network such as the Internet or an intranet via the communication interface 96.

The processor 91 develops a program (instruction) stored in the auxiliary storage device 93 or the like in the main storage device 92. For example, the program is a software program for executing the processing of each example embodiment. The processor 91 executes the program developed in the main storage device 92. The processor 91 executes the processing according to each example embodiment by executing the program.

The main storage device 92 has an area in which a program is developed. A program stored in the auxiliary storage device 93 or the like is developed in the main storage device 92 by the processor 91. The main storage device 92 is achieved by a volatile memory such as a dynamic random access memory (DRAM). A nonvolatile memory such as a magneto resistive random access memory (MRAM) may be configured/added as the main storage device 92.

The auxiliary storage device 93 stores various pieces of data such as programs. The auxiliary storage device 93 is achieved by a local disk such as a hard disk or a flash memory. Various pieces of data may be stored in the main storage device 92, and the auxiliary storage device 93 may be omitted.

The input/output interface 95 is an interface that connects the information processing device 90 and a peripheral device based on a standard or a specification. The communication interface 96 is an interface that connects to an external system or a device through a network such as the Internet or an intranet in accordance with a standard or a specification. The input/output interface 95 and the communication interface 96 may be shared as an interface connected to an external device.

An input device such as a keyboard, a mouse, or a touch panel may be connected to the information processing device 90 as necessary. These input devices are used to input of information and settings. When a touch panel is used as the input device, a screen having a touch panel function serves as an interface. The processor 91 and the input device are connected via the input/output interface 95.

The information processing device 90 may be provided with a display device that displays information. In a case where a display device is provided, the information processing device 90 includes a display control device (not illustrated) that controls display of the display device. The information processing device 90 and the display device are connected via the input/output interface 95.

The information processing device 90 may be provided with a drive device. The drive device mediates reading of data and a program stored in a recording medium and writing of a processing result by the information processing device 90 to the recording medium between the processor 91 and the recording medium (program recording medium). The information processing device 90 and the drive device are connected via an input/output interface 95.

The above is an example of a hardware configuration for enabling the process according to each example embodiment of the present invention. The hardware configuration of FIG. 20 is an example of a hardware configuration for executing the processing according to each example embodiment, and does not limit the scope of the present invention. A program for causing a computer to execute the process according to each example embodiment is also included in the scope of the present invention.

A program recording medium recording the program according to each example embodiment is also included in the scope of the present invention. The recording medium can be achieved by, for example, an optical recording medium such as a compact disc (CD) or a digital versatile disc (DVD). The recording medium may be achieved by a semiconductor recording medium such as a Universal Serial Bus (USB) memory or a secure digital (SD) card. The recording medium may be achieved by a magnetic recording medium such as a flexible disk, or another recording medium. In a case where the program executed by the processor is recorded in the recording medium, the recording medium is a program recording medium.

The components of the example embodiments may be combined in any manner. The components of the example embodiments may be implemented by software. The components of each example embodiment may be implemented by a circuit.

The previous description of embodiments is provided to enable a person skilled in the art to make and use the present invention. Moreover, various modifications to these example embodiments will be readily apparent to those skilled in the art, and the generic principles and specific examples defined herein may be applied to other embodiments without the use of inventive faculty. Therefore, the present invention is not intended to be limited to the example embodiments described herein but is to be accorded the widest scope as defined by the limitations of the claims and equivalents.

Further, it is noted that the inventor's intent is to retain all equivalents of the claimed invention even if the claims are amended during prosecution.

1. A gait information generation device comprising:
at least one memory storing instructions; and
at least one processor connected to the at least one memory and configured to execute the instructions to:
acquire gait data including time-series data of a foot position of a subject;
detect a start point and an end point of a gait cycle from the time-series data of the foot position included in the gait data;
calculate a first movement amount component related to the start point and a second movement amount component related to the end point;
generate gait information about a gait movement path of the subject using the calculated first movement amount component and the calculated second movement amount component; and
output the generated gait information.

2. the gait information generation device according to claim 1, wherein
the at least one processor is configured to execute the instructions to
calculate an acceleration vector or a velocity vector at the start point as the first movement amount component, and
calculate an acceleration vector or a velocity vector at the end point as the second movement amount component.

3. The gait information generation device according to claim 2, wherein
the at least one processor is configured to execute the instructions to
detect, as the start point, a preceding first heel strike of two consecutive heel strikes, and
detect, as the end point, a subsequent second heel strike of the two consecutive heel strikes,
calculate, in a horizontal plane, a position of an intersection between a first auxiliary straight line that is parallel to the first movement amount component and passes through the start point and a second auxiliary straight line that is parallel to the second movement amount component and passes through the end point, and
derive a curved line passing through the start point, the end point, and the intersection as the gait movement path.

4. The gait information generation device according to claim 3, wherein
the at least one processor is configured to execute the instructions to
derive a curved line having the start point, the end point, and the intersection as control points as the gait movement path.

5. The gait information generation device according to claim 3, wherein
the at least one processor is configured to execute the instructions to
derive a Bezier curved line having the start point, the end point, and the intersection as control points as the gait movement path.

6. The gait information generation device according to claim 3, wherein
the at least one processor is configured to execute the instructions to
derive a curved line passing through the start point, the end point, and the intersection as the gait movement path when the intersection is located inside a specific region in which a first circle centered around the start point with a distance between the start point and the end point as a radius and a second circle centered around the end point with a distance between the start point and the end point as a radius overlap with each other, and
derive a straight line connecting the start point and the end point as the gait movement path when the intersection is located outside the specific region.

7. The gait information generation device according to claim 1, wherein
the at least one processor is configured to execute the instructions to
generate the gait information in which visual information including the gait movement path is superimposed on a frame constituting a video indicating a gait state of the subject,
output the gait information related to the subject to a terminal device, and
display display-information about the visual information included in the gait information on a screen of the terminal device.

8. The gait information generation device according to claim 1, wherein
the at least one processor is configured to execute the instructions to
generate the gait information including visual information in which information related to an abnormality detected from at least one of the first movement amount component and the second movement amount component related to the subject is superimposed on a frame constituting a video indicating a gait state of the subject,
output the gait information related to the subject to a terminal device, and
display display-information about the visual information included in the gait information on a screen of the terminal device.

9. A gait information generation method executed by a computer, the method comprising:
acquiring gait data including time-series data of a foot position of a subject;
detecting a start point and an end point of a gait cycle from the time-series data of the foot position included in the gait data;
calculating a first movement amount component related to the start point and a second movement amount component related to the end point;
generating gait information about a gait movement path of the subject using the calculated first movement amount component and the calculated second movement amount component; and
outputting the generated gait information.

10. A non-transitory recording medium recording a program for causing a computer to execute:
acquiring gait data including time-series data of a foot position of a subject;
detecting a start point and an end point of a gait cycle from the time-series data of the foot position included in the gait data;
calculating a first movement amount component related to the start point and a second movement amount component related to the end point;
generating gait information about a gait movement path of the subject using the calculated first movement amount component and the calculated second movement amount component; and
outputting the generated gait information.
